(19) United States
(12) Patent Application Publication
(10) Pub. No.: US 2025/0029685 A1
(43) Pub. Date: Jan. 23, 2025

(54) ARTIFICIAL INTELLIGENCE DEVICE AND METHOD FOR RECOGNIZING STRUCTURAL FORMULA IMAGE

(71) Applicant: LG MANAGEMENT DEVELOPMENT INSTITUTE CO., LTD., Seoul (KR)

(72) Inventors: Yeonsik JO, Seoul (KR); Ahra JO, Seoul (KR); Soonyoung LEE, Seoul (KR); Changyoung PARK, Seoul (KR); Hormazabal RODRIGO, Seoul (KR); Taehoon KIM, Seoul (KR)

(21) Appl. No.: 18/904,003

(22) Filed: Oct. 1, 2024

Related U.S. Application Data
(63) Continuation of application No. PCT/KR2023/003770, filed on Mar. 22, 2023.

(30) Foreign Application Priority Data
Apr. 1, 2022 (KR) 10-2022-0041054

(51) Int. Cl. G16C 20/70; G06T 11/20; G06V 10/86; G16C 20/20; G16C 20/80

(52) U.S. Cl. CPC G16C 20/70; G06T 11/206; G06V 10/86; G16C 20/20; G16C 20/80

(57) ABSTRACT

An artificial intelligence device includes a memory in which a structural formula image is stored, and a processor configured to obtain information on a plurality of atomic regions from the structural formula image, to obtain information on bonding relationships between a plurality of atoms on the basis of the information on the plurality of atomic regions, to generate an adjacency matrix on the basis of the information on the plurality of atomic regions and the information on bonding relationships between the plurality of atoms, and to generate a predetermined string format corresponding to the structural formula image on the basis of the adjacency matrix.

### CROSS REFERENCE TO RELATED APPLICATIONS

This application is a continuation of International Application No. PCT/KR2023/003770, filed on Mar. 22, 2023, which claims the priority to Korean Patent Application No. 10-2022-0041054, filed on Apr. 1, 2022. The disclosures of the prior applications are considered part of the disclosure of this application and are hereby incorporated by reference in their entireties.

### BACKGROUND

### Field

The present disclosure generally relates to an artificial intelligence device and a method for recognizing a structural formula image. Specifically, the present disclosure relates to an artificial intelligence device and a method for recognizing a chemical structural formula image or a molecular structural formula image and converting the structural formula image into a predetermined string.

### Related Art

A structural formula may mean a graphically expressed chemical structure or molecular structure. The structural formula can show how atoms are arranged in a three-dimensional space. It can also clearly or implicitly display chemical bonding of molecules.

In particular, unlike a molecular formula that has a limited number of symbols and can only provide limited descriptions, the structural formula can provide geometric information of a molecular structure. For example, the structural formula can express isomers that have the same molecular formula but different structures or arrangements of atoms.

In particular, structural formulas may be provided in the form of images in various literature, papers, etc.

However, the structural formula images generally have a problem that makes searching difficult.

Therefore, there is a growing need to convert a structural formula image into a predetermined string to facilitate searching.

### SUMMARY

An object of the present disclosure is to solve the aforementioned problem and other problems.

An object of some embodiments of the present disclosure may be to provide an artificial intelligence device and method thereof capable of obtaining information on atoms from a structural formula image and more accurately recognizing bonding relationships between atoms.

An object of certain embodiments of the present disclosure may be to provide an artificial intelligence device and method thereof capable of recognizing a structural formula image and converting the structural formula image into a predetermined string format.

An embodiment of the present disclosure provides an artificial intelligence device including a memory in which a structural formula image is stored, and a processor that obtains information on a plurality of atomic regions from the structural formula image, obtains information on bonding relationships between a plurality of atoms on the basis of the information on the plurality of atomic regions, generates an adjacency matrix on the basis of the information on the plurality of atomic regions and the information on bonding relationships between the plurality of atoms, and generates a predetermined string format corresponding to the structural formula image on the basis of the adjacency matrix.

An embodiment of the present disclosure provides an artificial intelligence device including a processor that inputs a structural formula image to an atomic region recognition model and obtain information on a plurality of atomic regions output from the atomic region recognition model.

An embodiment of the present disclosure provides an artificial intelligence device including a processor that obtains a bonding image between a first atom and a second atom on the basis of information on a first atomic region and information on a second atomic region from among information on a plurality of atomic regions, and obtains information on a bonding relationship between the first atom and the second atom on the basis of the bonding image.

An embodiment of the present disclosure provides an artificial intelligence device including a processor that inputs a bonding image to a bonding relationship recognition model and obtains information on a bonding relationship output from the bonding relationship recognition model.

An embodiment of the present disclosure provides an artificial intelligence device including a processor that obtains a bonding image including a center point position of a first atomic region and a center point position of a second atomic region on the basis of information on the first atomic region and information on the second atomic region.

An embodiment of the present disclosure provides an artificial intelligence device including a processor that selects a second atomic region located within a predetermined distance on the basis of information on a first atomic region.

An embodiment of the present disclosure provides an artificial intelligence device including a processor that generates an adjacency matrix having a plurality of atoms as vertices and having information on bonding relationships between the plurality of atoms as edges on the basis of information on a plurality of atomic regions.

An embodiment of the present disclosure provides an artificial intelligence device including a processor that converts a structural formula image into a predetermined string format including a Simplified Molecular Input Line Entry System (SMILE) on the basis of a generated adjacency matrix.

According to an embodiment of the present disclosure, an artificial intelligence device can obtain information on atoms from a structural formula image and more accurately recognize bonding relationships between atoms.

According to an embodiment of the present disclosure, an artificial intelligence device can recognize a structural formula image and convert the sane into a predetermined string format.

### BRIEF DESCRIPTION OF THE DRAWINGS

FIG. 1 is a block diagram for illustrating an artificial intelligence device according to an embodiment of the present disclosure.

FIG. 2 is a block diagram for illustrating an artificial intelligence server according to an embodiment of the present disclosure.

FIG. 3 is a flowchart illustrating a structural formula recognition method according to an embodiment of the present disclosure.

FIG. 4 is a diagram illustrating an example of a structural formula image according to an embodiment of the present disclosure.

FIG. 5 is a diagram illustrating an atomic region recognition model according to an embodiment of the present disclosure.

FIG. 6 is a diagram illustrating an example of information on a plurality of atomic regions according to an embodiment of the present disclosure.

FIG. 7 is a diagram illustrating a method for obtaining bonding relationship information according to an embodiment of the present disclosure.

FIG. 8 is a diagram illustrating a bonding relationship recognition model according to an embodiment of the present disclosure.

FIG. 9 is a diagram illustrating an example of an adjacency matrix according to one embodiment of the present disclosure.

### DESCRIPTION OF EXEMPLARY EMBODIMENTS

Hereinafter, embodiments of the present disclosure will be described in detail with reference to the attached drawings, and identical or similar components will be given the same reference numerals and redundant descriptions thereof will be omitted regardless of the drawing symbols. The suffixes “module” and “unit” of elements herein are used for convenience of description and thus can be used interchangeably and do not have any distinguishable meanings or functions. In the following description of embodiments of the present disclosure, a detailed description of known functions and configurations incorporated herein will be omitted when it may obscure the subject matter of the present disclosure. The same reference numbers will be used throughout this specification to refer to the same or like parts. In addition, the attached drawings are only intended to facilitate easy understanding of the embodiments of the present disclosure, and the technical ideas disclosed in the present disclosure are not limited by the attached drawings and should be understood to include all modifications, equivalents, or substitutes included in the spirit and technical scope of the present disclosure.

Terms including ordinal numbers, such as first, second, etc., may be used to describe various components, but the components are not limited by the terms. The terms are used only to distinguish one component from another.

When a component is referred to as being “coupled” or “connected” to another component, it should be understood that the component may be directly or indirectly coupled or connected to the other component and therefore there may be other components therebetween. On the other hand, when a component is referred to as being “directly coupled” or “directly connected” to another component, it should be understood that there are no other components therebetween.

### Artificial Intelligence (AI)

Artificial intelligence may refer to a field in which artificial intelligence or a methodology for creating the artificial intelligence is studied, and machine learning may refer to a field in which various problems in the field of artificial intelligence are defined and a methodology for solving the various problems in the field of artificial intelligence is studied. The machine learning may be also defined as an algorithm of improving the performance of a task through constant experience.

An artificial neural network (ANN) is a model used in the machine learning and can refer to an entire model having a problem-solving ability and comprising artificial neurons (nodes) constituting a network by combining synapses. An artificial neural network can be defined by a connection pattern between neurons in different layers, a learning process of updating model parameters, and an activation function of generating output values.

An artificial neural network can include an input layer and an output layer. Optionally, the artificial neural network may further comprise one or more hidden layers. Each layer may include one or more neurons, and the artificial neural network can include synapses that connect neurons. In the artificial neural network, each neuron can output a function value of an activation function for input signals, weights, and biases input through synapses.

Model parameters may refer to parameters determined through learning and include weights of synaptic connections and bias of neurons. In addition, hyper-parameters refer to parameters that need to be set before learning in machine learning algorithms and include a learning rate, the number of repetitions, a mini-batch size, an initialization function, and the like.

The purpose of the learning of the artificial neural network can be regarded as determining model parameters that reduce or minimize a loss function. The loss function can be used as an index for determining optimal model parameters during a learning process of the artificial neural network.

The machine learning can be classified into supervised learning, unsupervised learning, and reinforcement learning depending on the learning method.

The supervised learning refers to a method of training an artificial neural network when labels for training data are given, and the labels refer to a correct answer (or result value) that the artificial neural network needs to infer when training data is input to the artificial neural network. Unsupervised learning refers to a method of training an artificial neural network when labels for training data are not given. Reinforcement learning refers to a learning method of training an agent defined in a certain environment such that the agent selects an action or an action sequence that maximizes a cumulative reward in each state.

Among artificial neural networks, machine learning implemented with a deep neural network (DNN) including a plurality of hidden layers is called deep learning, and the deep learning is a part of machine learning. Hereinafter, it is assumed that the machine learning includes the deep learning.

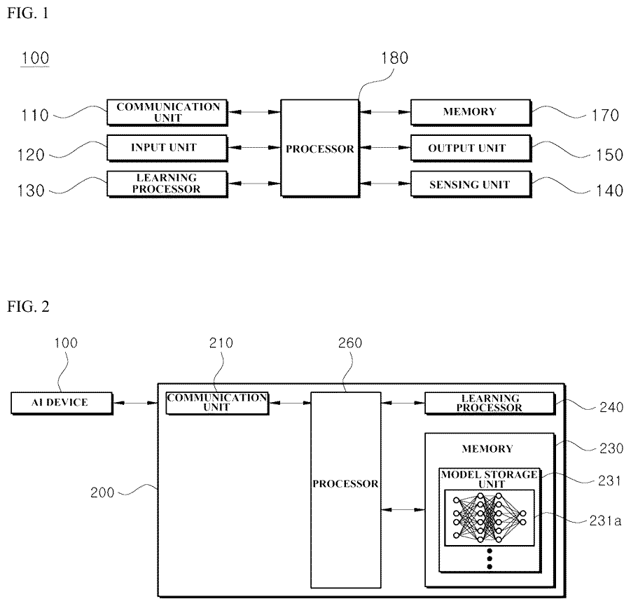

FIG. 1 is a block diagram for illustrating an artificial intelligence device according to an embodiment of the present disclosure.

The artificial intelligence (AI) device 100 may be implemented as or included in a stationary device or a movable device, such as a TV, a projector, a mobile phone, a smartphone, a desktop computer, a laptop computer, a digital broadcasting terminal, a personal digital assistant (PDA), a portable multimedia player (PMP), a navigation device, a tablet PC, a wearable device, a set-top box (STB), a DMB receiver, a radio receiver, a washing machine, a refrigerator, digital signage, a robot, a vehicle, or the like.

Referring to FIG. 1, the AI device 100 may include a communication unit or communicator 110, an input unit 120, a learning processor 130, a sensing unit 140, an output unit 150, a memory 170, and a processor 180.

The communication unit 110 may transmit and receive data to and from external devices such as another or other AI devices and an AI server 200 using wired and wireless communication technology. For example, the communication unit 110 may transmit and receive sensor information, user input, trained models, control signals, etc. to and from the external devices.

Here, the communication technologies used by the communication unit 110 include, for example, but not limited to, Global System for Mobile communication (GSM), Code Division Multi Access (CDMA), Long Term Evolution (LTE), 5G, wireless LAN (WLAN), Wireless-Fidelity (Wi-Fi), Bluetooth, Radio Frequency Identification (RFID), Infrared Data Association (IrDA), ZigBee, Near Field Communication (NFC), etc.

The input unit 120 may obtain various types of data. Here, the input unit 120 may include, for instance, but not limited to, a camera for video signal input, a microphone for receiving audio signals, a user input unit for receiving information from a user, etc. Here, the camera or the microphone may perform a function of a sensor, and a signal obtained from the camera or the microphone may be referred to as sensing data or sensor information.

The input unit 120 may obtain training data for model training and input data to be used when output is obtained using a trained model. The input unit 120 may obtain unprocessed input data, and in this case, the processor 180 or the learning processor 130 may extract input features as pre-processing for the input data.

The learning processor 130 may train a model configured using an artificial neural network using training data. Here, the trained artificial neural network may be called a trained model. The trained model may be used to infer result values for new input data other than training data, and the inferred values may be used as a basis for determination for performing a certain operation.

Here, the learning processor 130 may be configured to perform AI processing along with a learning processor 240 of the AI server 200.

The learning processor 130 may include a memory integrated or implemented in the AI device 100. Alternatively, the learning processor 130 may be implemented using a memory 170, an external memory directly or indirectly coupled to the AI device 100, or a memory included in an external device.

The sensing unit 140 may obtain at least one of internal information of the AI device 100, information on the surrounding environment of the AI device 100, and user information using various sensors. The sensing unit 140 may include one or more sensors.

Here, sensors included in the sensing unit 140 include a proximity sensor, an illuminance sensor, an acceleration sensor, a magnetic sensor, a gyro sensor, an inertial sensor, a visible imaging sensor such as a red-green-blue (RGB) sensor, an infrared (IR) sensor, a fingerprint recognition sensor, an ultrasonic sensor, a light sensor, a microphone, a lidar, a radar, etc.

The output unit 150 may generate visual, auditory, or tactile output.

The output unit 150 may include a display unit configured to output visual information, a speaker configured to output auditory information, a haptic module configured to output tactile information, etc.

The memory 170 may store data that supports various functions of the AI device 100. For example, the memory 170 may store data, instructions, models such as input data, training data, trained models, learning history, etc. acquired through the input unit 120.

The processor 180 may determine at least one executable operation of the AI device 100 on the basis of information determined or generated using a data analysis algorithm or a machine learning algorithm. In addition, the processor 180 may control components of the AI device 100 to perform a determined operation.

To this end, the processor 180 may request, retrieve, receive or utilize data from the learning processor 130 or the memory 170 and control components of the AI device 100 to execute a predicted operation or an operation determined to be desirable among the at least one executable operation.

Here, if an external device is required to be connected to the AI device 100 to perform a determined operation, the processor 180 may generate a control signal for controlling the external device and transmit the generated control signal to the external device.

The processor 180 may obtain intention information with respect to user input and determine user's requirement based on the obtained intention information.

Here, the processor 180 may obtain intention information corresponding to user input by using at least one of a Speech To Text (STT) engine for converting audio input into a string or a natural language processing (NLP) engine for obtaining intention information of a natural language.

At least a part of at least one of the STT engine or the NLP engine may be configured as an artificial neural network trained according to a machine learning algorithm. In addition, at least one of the STT engine or the NLP engine may be trained by the learning processor 130, trained by the learning processor 240 of the AI server 200, or trained through distributed processing of the learning processor 130 and the learning processor 240.

The processor 180 may collect history information including the operation details of the AI device 100 or user's feedback for operations and store the history information in the memory 170 or the learning processor 130 or transmit the history information to an external device such as an AI server 200. The collected history information may be used to update a trained model.

The processor 180 may control one or some of the components of the AI device 100 to operate an application program stored in the memory 170. Furthermore, the processor 180 may operate two or more of the components included in the AI device 100 in combination in order to operate the application program.

FIG. 2 is a block diagram for illustrating an artificial intelligence server according to an embodiment of the present disclosure.

Referring to FIG. 2, the AI server 200 may be a device configured to train an artificial neural network using a machine learning algorithm or use a trained artificial neural network. For instance, the AI server 200 may comprise a plurality of servers to perform distributed processing or may be defined as a 5G network. In this case, the AI server 200 may be included as a part of the AI device 100 to perform at least some of AI processing along with the AI device 100.

The AI server 200 may include a communication unit 210, a memory 230, the learning processor 240, a processor 260, and so on.

The communication unit or communicator 210 may transmit and receive data to and from an external device such as the AI device 100.

The memory 230 may include a model storage unit 231. The model storage unit 231 may store a model (or artificial neural network 231a) that is being trained or has been trained through the learning processor 240.

The learning processor 240 may train the artificial neural network 231a using training data. The trained model may be used while being loaded into the AI server 200 of the artificial neural network or may be used by being loaded into an external device such as the AI device 100.

The trained model may be implemented as hardware, software, or combination of hardware and software. If part or whole of the trained model is implemented as software, one or more instructions constituting the trained model may be stored in the memory 230.

The processor 260 may infer result values for new input data using the trained model and generate a response or control command on the basis of the inferred result values.

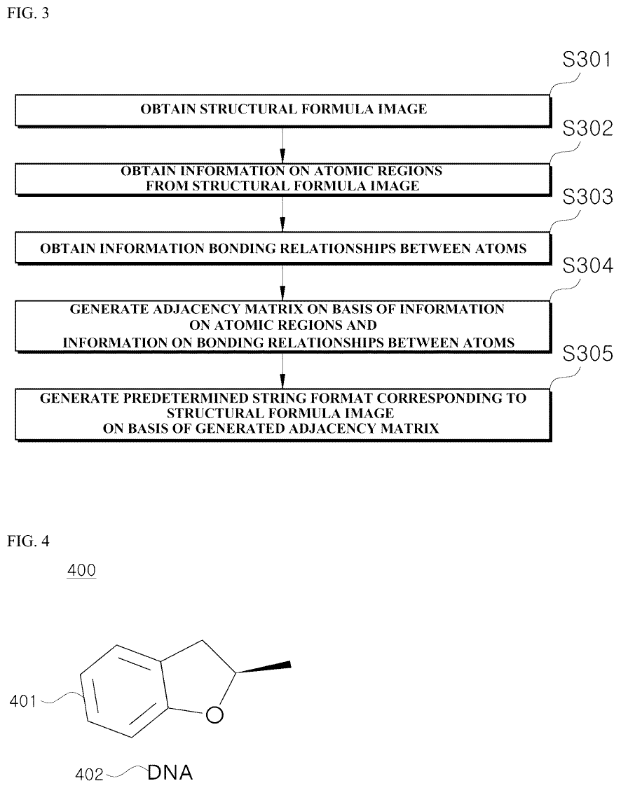

FIG. 3 is a flowchart illustrating a structural formula recognition method according to an embodiment of the present disclosure.

The processor 180 may obtain a structural formula image (operation S301). The structural formula image may be an image stored in the memory 170 or an image received from an external device through the communication unit 110.

The structural formula image may be an image in which a structural formula is expressed in a graphical form.

The structural formula may refer to a graphical representation of a chemical structure or a molecular structure. The structural formula may include information on the arrangement of atoms in a three-dimensional space. In addition, the structural formula may also express chemical bonding between atoms.

Meanwhile, a structural formula image may include annotations. For example, annotations may mean the names of compounds.

FIG. 4 is a diagram illustrating an example of a structural formula image according to an embodiment of the present disclosure.

A structural formula image 400 may include a structural formula 401 that graphically expresses a molecular structure. In addition, the structural formula image 400 may include an annotation 402. When the structural formula image 400 is generated, an annotation 402 may be imaged and included in the structural formula image 400 as the description or name of the molecular structure. Therefore, in a case where the structural formula image 400 is recognized and converted into a predetermined string format, it is necessary to filter only the structural formula 401 as a recognition target and exclude the annotation 402 included in the structural formula image 400.

Referring back to FIG. 3, the processor 180 may obtain information on a plurality of atomic regions from the structural formula image (operation S302). The atomic region information may include at least one of information on an atomic region identification, information on an atomic position, and information on one or more atoms in the structural formula image.

The information on an atomic region identification or atomic region identification information may be identification information (such as a number) for identifying each of a plurality of atomic regions recognized in the structural formula image.

In addition, the information on an atomic position or atomic position information may be coordinate information corresponding to an atomic region in the structural formula image. For example, when an atomic region is expressed as a square, the atomic position information may include the coordinates of each vertex of the square corresponding to the atomic region and the coordinates of the center point of the square.

In addition, the information on an atom or atomic information may include element symbol information of an atom corresponding to an atomic region. For example, if an image corresponding to an atomic region is expressed as a vertex, carbon (C) may correspond to atomic information. In addition, if an element symbol (for example, oxygen (O)) is written in an image corresponding to an atomic region, information on the element symbol (O) may correspond to atomic information.

Meanwhile, the information on an atomic region or atomic region information may include reliability information of atomic region information acquired from the structural formula image. For example, the processor 180 may acquire reliability information of the atomic region information acquired from the structural formula image as a value between 0.00 and 1.00 and may use only atomic region information having reliability information equal to or greater than a predetermined value.

Meanwhile, the processor 180 may input the structural formula image to an atomic region recognition model and may acquire information on a plurality of atomic regions output from the atomic region recognition model.

FIG. 5 is a diagram illustrating an atomic region recognition model according to an embodiment of the present disclosure.

Referring to FIG. 5, the processor 180 may input a structural formula image 501 to an atomic region recognition model 502, and obtain atomic region information output from the atomic region recognition model 502.

The atomic region recognition model 502 may be an artificial neural network (ANN) trained to output at least one piece of atomic region information 503 included in an image for the input structural formula image 501. The ANN is a model used in machine learning and may refer to a model having a problem-solving ability and comprising artificial neurons (nodes) that constitute a network by combining synapses. For example, the atomic region recognition model 502 may be an artificial neural network model based on convolutional neural networks (CNN).

The learning processor 130 may train the atomic region recognition model 502 having an artificial neural network using training data on structural formula images and atomic region information. Meanwhile, the atomic region recognition model 502 may be a model trained by the learning processor 240 of the AI server 200.

The trained atomic region recognition model 502 may be stored in the memory 170 or in the model storage unit 231 of the AI server 200. The processor 180 may use the atomic region recognition model 502 stored in the memory 170 or the model storage unit 231.

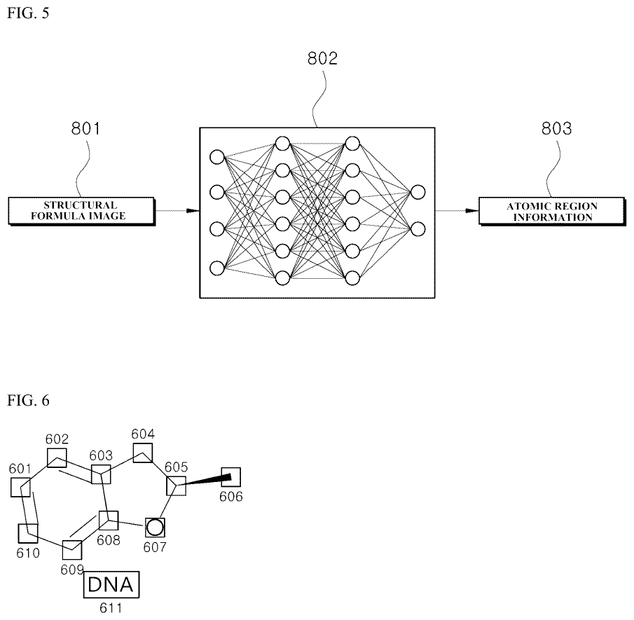

FIG. 6 is a diagram illustrating an example of information on a plurality of atomic regions according to an embodiment of the present disclosure.

Referring to FIG. 6, the processor 180 may obtain the information on a plurality of atomic regions 601, 602, 603, 604, 605, 606, 607, 608, 609, 610, and 611, output from the atomic region recognition model 502.

The information on the plurality of atomic regions may include information on vertex regions 601, 602, 603, 604, 605, 606, 608, 609, and 610, an element symbol region 607 in which an element symbol is written, and an annotation region 611 in the structural formula. For example, since the atomic region recognition model 502 can be trained to output the element symbol region 607 in which the element symbol is written, it may be possible that the atomic region recognition model 502 outputs the annotation region 611 as atomic region information. Therefore, it is necessary to filter information on the annotation region from the information on the plurality of atomic regions. For example, a bonding relationship with other atomic regions can be considered to classify an annotation. Since the annotation region 611 does not have a bonding relationship with other atomic regions, the processor 180 may classify the annotation region 611 as an annotation if there is no bonding relationship between the annotation region 611 and other atomic regions.

Referring back to FIG. 3, the processor 180 may obtain information on bonding relationships between a plurality of atoms based on the information on the plurality of atomic regions (operation S303).

The processor 180 may obtain information on bonding relationships between other atomic regions for each atomic region based on the information on the plurality of atomic regions.

The processor 180 may obtain a bonding image between a first atom and a second atom on the basis of first atomic region information and second atomic region information among the information on the plurality of atomic regions, and may obtain information on the bonding relationship between the first atom and the second atom on the basis of the obtained bonding image.

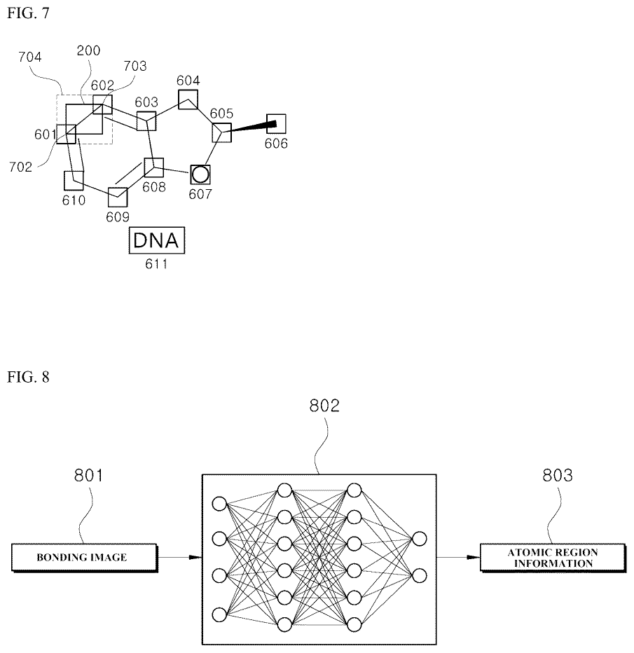

FIG. 7 is a diagram illustrating a method for obtaining bonding relationship formation according to an embodiment of the present disclosure.

The processor 180 may select first atomic region information (information on a first atomic region) 601 from among the information on the plurality of atomic regions. In addition, the processor 180 may select second atomic region information (information on a second region) 602 that is different from the first atomic region information. Further, the processor 180 may obtain an image of a bond between two atoms such as a bonding image 704 between the first atom and the second atom on the basis of first atomic position information 702 of the first atomic region information 601 and second atomic position information 703 of the second atomic region information 602. For example, the first atomic position information 702 and the second atomic position information 703 may be the center point positions of the atomic regions. However, they are not limited to the center point positions of the atomic regions, and can be any point position associated with an atomic region if necessary.

The processor 180 may obtain the bonding image 704 including the center point positions of the atomic regions on the basis of the first atomic position information 702 of the first atomic region information 601 and the second atomic position information 703 of the second atomic region information 602.

In addition, the processor 180 may obtain the bonding image 704 including the first atomic region and the second atomic region on the basis of the first atomic region information 601 and the second atomic region information 602. The size and shape of the bonding image may be adjusted in various ways.

Further, the processor 180 may obtain a bonding image between atoms that can be combined for each of the plurality of atomic regions. However, if bonding images between all atoms that can be combined are obtained, the amount of computation may increase. Therefore, the processor 180 may obtain only a bonding image between the first atom and the second atom, which are located within a predetermined distance from each other, on the basis of the information on the plurality of atomic regions.

For example, the processor 180 may select a second atomic region located within a predetermined distance from a first atomic region on the basis of the first atomic region information among the information on the plurality of atomic regions. Referring to FIG. 7, the processor 180 may identify second atomic regions 602, 603, 608, 609, and 610 located within a predetermined distance from the first atomic region 601, obtain a bonding image between the first atom region 601 and each of the identified second atom regions 602, 603, 608, 609, and 610, and obtain information on bonding relationships between the first atom and the second atoms. In addition, the processor 180 may determine that third atomic regions 604, 605, 606, 607, and 611 located outside a predetermined distance from the first atomic region 601 do not have bonding relationships. Therefore, the amount of computation can be reduced.

Further, the processor 180 may obtain information on bonding relationships between atoms on the basis of acquired bonding images.

For example, the processor 180 may input the bonding images to a bonding relationship recognition model and obtain bonding relationship information output from the bonding relationship recognition model.

FIG. 8 is a diagram illustrating a bonding relationship recognition model according to an embodiment of the present disclosure.

Referring to FIG. 8, the processor 180 may input a bonding image 801 to a bonding relationship recognition model 802 and obtain bonding relationship information output from the bonding relationship recognition model 802.

The bonding relationship information is information on bonding between atoms and may include information on non-bonding, single bonding, double bonding, triple bonding, upward bonding, and downward bonding. The non-bonding may mean a case where there is no bonding between atoms.

The upward bonding may mean forward bonding on a plane indicated by a wedge. The downward bonding may mean backward bonding from a plane indicated by a dash.

The bonding relationship recognition model 802 may be an artificial neural network (ANN) trained to output bonding relationship information 803 for the input bonding image 801. The ANN is a model used in machine learning and may refer to a model having problem-solving ability and comprising artificial neurons (nodes) constituting a network by combining synapses. For example, the bonding relationship recognition model 802 may be an artificial neural network model based on the CNN.

The learning processor 130 may train the bonding relationship recognition model 802 configured as an artificial neural network using training data regarding bonding images and bonding relationship information. Meanwhile, the bonding relationship recognition model 802 may be a model trained by the learning processor 240 of the AI server 200.

The trained bonding relationship recognition model 802 may be stored in the memory 170 or in the model storage unit 231 of the AI server 200. The processor 180 may use the bonding relationship recognition model 802 stored in the memory 170 or the model storage unit 231.

Referring back to FIG. 3, the processor 180 may generate an adjacency matrix on the basis of the information on the plurality of atomic regions and the information on bonding relationships between the plurality of atoms (operation S304).

The processor 180 may generate an adjacency matrix using each of the plurality of atoms as a vertex and using bonding relationship information of each of the plurality of atoms as an edge.

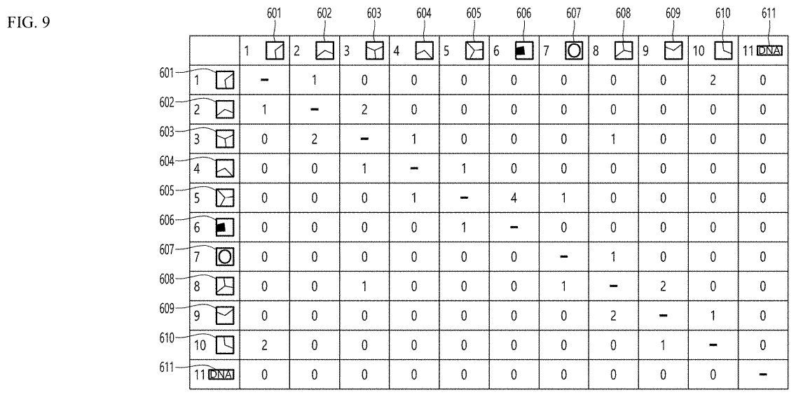

FIG. 9 is a diagram illustrating an example of an adjacency matrix according to one embodiment of the present disclosure.

Referring to FIG. 9, the processor 180 may generate the atoms of the plurality of atomic regions 601 to 611 as vertices of an adjacent matrix. Further, the processor 180 may generate the adjacent matrix using bonding relationship information of each of the plurality of atoms as an edge. For example, the processor 180 may generate an edge value of the adjacency matrix by setting each piece of bonding relationship information to an arbitrary number. For example, the processor 178 may set non-bonding to “0”, set single bonding to “1”, set double bonding to “2”, and set triple bonding to “3”.

In addition, the processor 180 may set upward bonding to “5” in the case of upward bonding in the order of a row and a column with the row as a starting vertex and the column as an arrival vertex and to “0” in the case of the opposite direction in order to indicate directionality. For example, referring to FIG. 9, the value of the sixth column of the fifth row of the adjacency matrix may be generated as a value of “5” due to upward bonding from the first atom 605 to the second atom 606. On the other hand, the value of the fifth column of the sixth row of the adjacency matrix in the opposite direction may be generated as “0”.

Similarly, the processor 180 may set downward bonding to “6” in the case of downward bonding in the order of a row and a column with the row as a starting vertex and the column as an arrival vertex and to “0” in the case of the opposite direction in order to indicate directionality.

Referring back to FIG. 3, the processor 180 may generate a predetermined string format corresponding to the structural formula image on the basis of the generated adjacency matrix (operation S305).

The processor 180 may obtain information on bonding relationships with other atoms by traversing the atomic regions corresponding to the vertices of the adjacency matrix. The processor 180 may identify atom information of each of the plurality of atomic regions using the obtained bonding relationship information between atoms and generate a predetermined string format corresponding to the structural formula image on the basis of the plurality of pieces of atomic information and the information on bonding relationships between the plurality of atoms.

In this case, the string format is a file format that can represent information of compounds (e.g., element positions, bonding relationships, etc.) and may include a MOL file format, an SDF file format, or the like. Meanwhile, the string format may include information on Simplified Molecular Input Line Entry System (SMILES).

The above-described present disclosure can be implemented as computer-readable code on a medium in which a program is recorded. Computer-readable media include all types of recording devices in which data readable by a computer system is stored.

Examples of computer-readable media include a hard disk drive (HDD), a solid state drive (SSD), a silicon disk drive (SDD), a ROM, a RAM, a CD-ROM, a magnetic tape, a floppy disc, an optical data storage device, etc. In addition, the computer may include the processor 180 of the AI device 100.

What is claimed is:

1. An artificial intelligence device comprising:
a memory configured to store a structural formula image; and
a processor configured to obtain information on a plurality of atomic regions from the structural formula image, obtain information on bonding relationships between a plurality of atoms based on the information on the plurality of atomic regions, generate an adjacency matrix based on the information on the plurality of atomic regions and the information on the bonding relationships between the plurality of atoms, and generate a string format corresponding to the structural formula image based on the adjacency matrix.

2. The artificial intelligence device of claim 1, wherein the processor is configured to input the structural formula image to an atomic region recognition model and obtain the information on the plurality of atomic regions output from the atomic region recognition model.

3. The artificial intelligence device of claim 2, wherein the information on the plurality of atomic regions includes information on positions of the plurality of atomic regions and information on the plurality of atoms.

4. The artificial intelligence device of claim 3, wherein the processor is configured to obtain a bonding image between a first atom and a second atom based on information on a first atomic region and information on a second atomic region from the information on the plurality of atomic regions, and obtain information on a bonding relationship between the first atom and the second atom based on the bonding image.

5. The artificial intelligence device of claim 4, wherein the processor is configured to input the bonding image to a bonding relationship recognition model, and obtain the information on the bonding relationship output from the bonding relationship recognition model.

6. The artificial intelligence device of claim 4, wherein the processor is configured to obtain information on a position of a center point of the first atomic region and a position of a center point of the second atomic region based on the information on the first atomic region and the information on the second atomic region.

7. The artificial intelligence device of claim 4, wherein the processor is configured to select the second atomic region located within a predetermined distance from the first atomic region.

8. The artificial intelligence device of claim 1, wherein the processor is configured to generate the adjacency matrix having the plurality of atoms as vertices and having the information on bonding relationships between the plurality of atoms as edges based on the information on the plurality of atomic regions.

9. The artificial intelligence device of claim 1, wherein the processor is configured to convert the structural formula image into the string format including a Simplified Molecular Input Line Entry System (SMILE) based on the generated adjacency matrix.

10. A computerized method comprising:
obtaining information on a plurality of atomic regions from a structural formula image;
obtaining information on bonding relationships between a plurality of atoms based on the information on the plurality of atomic regions;
generating an adjacency matrix based on the information on the plurality of atomic regions and the information on the bonding relationships between the plurality of atoms; and
generating a string format corresponding to the structural formula image based on the adjacency matrix.

11. The computerized method of claim 10, wherein the obtaining of the information on the plurality of atomic regions comprises:
inputting the structural formula image to an atomic region recognition model; and
obtaining the information on the plurality of atomic regions output from the atomic region recognition model,
wherein the information on the plurality of atomic regions includes information on positions of the plurality of atomic regions and information on the plurality of atoms.

12. The computerized method of claim 11, wherein the obtaining of the information on the bonding relationships comprises:
obtaining a bonding image between a first atom and a second atom based on information on a first atomic region and information on a second atomic region from the information on the plurality of atomic regions; and
obtaining information on a bonding relationship between the first atom and the second atom based on the bonding image,
wherein the obtaining of the information on the bonding relationship comprises:
inputting the bonding image to a bonding relationship recognition model; and
obtaining the information on the bonding relationship output from the bonding relationship recognition model.

13. The computerized method of claim 12, further comprising:
obtaining information on a position of a center point of the first atomic region and a position of a center point the second atomic region based on the information on the first atomic region and the information on the second atomic region; and
selecting the second atomic region located within a predetermined distance from the first atomic region.

14. The computerized method of claim 10, wherein the generating of the adjacency matrix comprises generating the adjacency matrix having the plurality of atoms as vertices and having the information on bonding relationships between the plurality of atoms as edges based on the information on the plurality of atomic regions.

15. The computerized method of claim 10, wherein the generating of the string format comprises converting the structural formula image into the string format including a Simplified Molecular Input Line Entry System (SMILE) based on the generated adjacency matrix.

16. A non-transitory computer-readable medium configured to store instructions that when executed by one or more processors, cause the one or more processors to perform operations comprising:
obtaining information on a plurality of atomic regions from a structural formula image;
obtaining information on bonding relationships between a plurality of atoms based on the information on the plurality of atomic regions;
generating an adjacency matrix based on the information on the plurality of atomic regions and the information on the bonding relationships between the plurality of atoms; and
generating a string format corresponding to the structural formula image based on the adjacency matrix.

17. The non-transitory computer-readable medium of claim 16, wherein the obtaining of the information on the plurality of atomic regions comprises:
inputting the structural formula image to an atomic region recognition model; and
obtaining the information on the plurality of atomic regions output from the atomic region recognition model,
wherein the information on the plurality of atomic regions includes information on positions of the plurality of atomic regions and information on the plurality of atoms.

18. The non-transitory computer-readable medium of claim 17, wherein the obtaining of the information on the bonding relationships comprises:
obtaining a bonding image between a first atom and a second atom based on information on a first atomic region and information on a second atomic region from the information on the plurality of atomic regions; and
obtaining information on a bonding relationship between the first atom and the second atom based on the bonding image,
wherein the obtaining of the information on the bonding relationship comprises:
inputting the bonding image to a bonding relationship recognition model; and
obtaining the information on the bonding relationship output from the bonding relationship recognition model.

19. The non-transitory computer-readable medium of claim 18, further comprising:
obtaining information on a position of a center point of the first atomic region and a position of a center point the second atomic region based on the information on the first atomic region and the information on the second atomic region; and
selecting the second atomic region located within a predetermined distance from the first atomic region.

20. The non-transitory computer-readable medium of claim 16, wherein the generating of the adjacency matrix comprises generating the adjacency matrix having the plurality of atoms as vertices and having the information on bonding relationships between the plurality of atoms as edges based on the information on the plurality of atomic regions.
